(19) United States
(12) Patent Application Publication
(10) Pub. No.: US 2023/0100914 A1
(43) Pub. Date: Mar. 30, 2023

(54) METHOD FOR OPERATION OF AN INDUSTRIAL PLANT AND AN INDUSTRIAL PLANT

(71) Applicant: Siemens Energy Global GmbH & Co. KG, Munich, Bayern (DE)

(72) Inventor: Stanislav Kubis, Ricany (CZ)

(73) Assignee: Siemens Energy Global GmbH & Co. KG, Munich, Bayern (DE)

(21) Appl. No.: 17/798,158

(22) PCT Filed: Dec. 17, 2020
(86) PCT No.: PCT/EP2020/086641

§ 371 Date: Aug. 8, 2022

(30) Foreign Application Priority Data
Feb. 14, 2020 (EP) 20157392.0

(51) Int. Cl. C10L 3/08; C25B 1/04; C25B 15/08; F02B 43/12; F23D 14/28; F23D 14/32

(52) U.S. Cl. CPC C10L 3/08; C25B 1/04; C25B 15/081; F02B 43/12; F23D 14/28; F23D 14/32; C10L 2290/02; C10L 2290/06; C10L 2290/38; F02B 2043/103

(57) ABSTRACT

A method for operation of an industrial plant having an energy accumulator unit for production of synthetic natural gas, a power plant unit for production of electricity, an oxygen tank, a carbon dioxide tank and a water tank. In a first operation mode the energy accumulator unit is supplied with excessed electricity from the public grid to produce synthetic natural gas, wherein the produced synthetic natural gas is discharged in a gas network, while oxygen and water which are produced together with the synthetic natural gas are stored in the oxygen tank and the water tank correspondingly. In a second operation mode gas from the gas network together with oxygen from the oxygen tank and water from the water tank are used in the power plant unit to produce electricity, which is supplied to the public grid. This way electricity production excess is efficiently accumulated for industrial or municipal use.

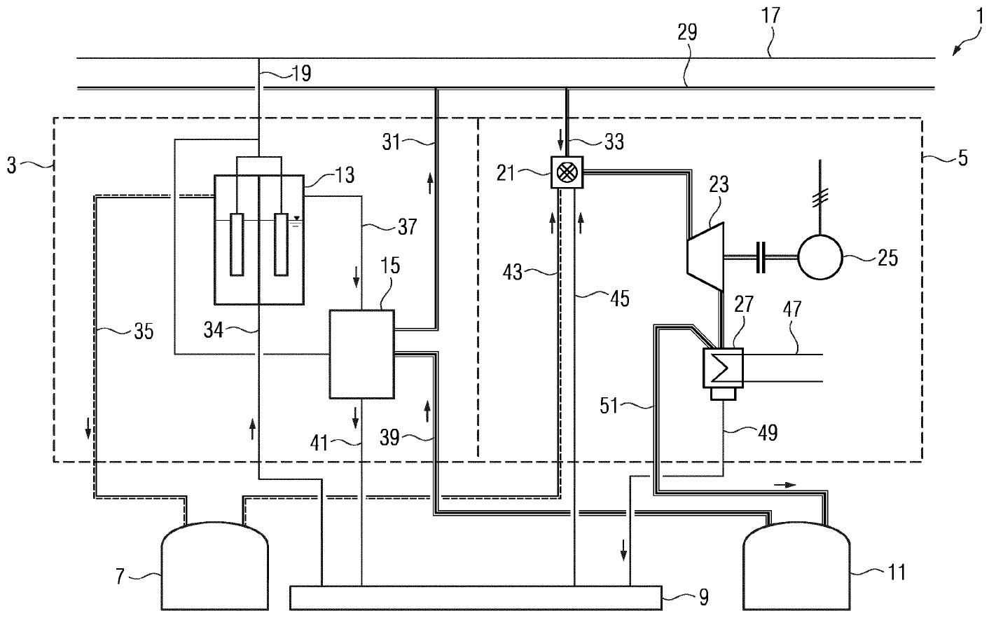

### CROSS REFERENCE TO RELATED APPLICATIONS

This application is the US National Stage of International Application No. PCT/EP2020/086641 filed 17 Dec. 2020, and claims the benefit thereof. The International Application claims the benefit of European Application No. EP20157392 filed 14 Feb. 2020. All of the applications are incorporated by reference herein in their entirety.

### FIELD OF INVENTION

The present invention relates to a method for operation of an industrial plant and such industrial plant.

### BACKGROUND OF INVENTION

The reduction of carbon dioxide emitted into the atmosphere on the one hand and increasing the share of renewable on the other hand cause some problems. One of the problems is how to use or store the electricity excess from the renewable sources, since the amount of produced power cannot be effectively controlled. Due to this fact, there is energy generated during times when there is low or no consumption for it. This surplus of electricity is currently accumulated in different kinds of energy accumulators such as pumped water storage plant, underground pneumatic accumulators, electric accumulators, etc.

Recently, new methods of energy storage have been applied at industrial scale. The electrical energy can be accumulated or stored by converting it into chemical energy of synthetic fuels such as hydrogen, methane or synthetic natural gas (SNG). In the prior art this process is referred to as “power to gas” technology.

One of the emerging technologies to accumulate the electricity excess is through the production of synthetic natural gas (SNG). The technology is based on the so-called Sabatier reaction, in which synthetic natural gas and water are formed from carbon dioxide (CO₂) and hydrogen (H₂) in the presence of catalysts. In the published studies the Carbon dioxide is obtained from biogas or coal while the hydrogen is obtained by electrolysis of water. Unfortunately, both substances biogas and coal contain certain additives. These additives play the role of impurities in the Sabatier reaction and contaminate the catalyst. This contamination reduces the efficiency of the Sabatier reaction until the reaction is not effective and the reactor has to be shutdown to recover the catalyst. It is necessary to transport these reactants for the Sabatier reaction from distant sites to the synthetic natural gas production site. Hence, there are two essential disadvantages of the above referred method: on the one hand the necessity to transport carbon dioxide or oxygen from distant locations and on the other hand the impurities in the carbon dioxide caused by the fuel additives.

### SUMMARY OF INVENTION

It is object of the present invention to provide an efficient process of accumulating the electricity surplus, which process eliminates the above-identified disadvantages.

The object of the invention is achieved by the independent claims. The dependent claims describe advantageous developments and modifications of the invention.

In accordance with the invention there is provided a method for operation of an industrial plant comprising:
an energy accumulator unit for production of synthetic natural gas,
a power plant unit for production of electricity,
an oxygen tank, a carbon dioxide tank and a water tank,
wherein
in a first operation mode the energy accumulator unit is supplied with excessed electricity from the public grid in order to produce synthetic natural gas, wherein the produced synthetic natural gas is discharged in a gas network, while oxygen and water which are produced together with the synthetic natural gas are stored in the oxygen tank and the water tank correspondingly,
in a second operation mode gas from the gas network together with oxygen from the oxygen tank and water from the water tank are used in the power plant unit to produce electricity, which is supplied to the public grid.

In accordance with the present invention there is provided also an industrial plant eligible for carrying out such operation method, the industrial plant comprising:
an energy accumulator unit for production of synthetic natural gas,
a power plant unit for production of electricity,
an oxygen tank, a carbon dioxide tank and a water tank.

Advantages relating to the described method may as well pertain to the industrial plant and vice versa.

The essential idea of the present invention is to connect the industrial plant to the public grid which transfers easily surplus renewable energy from distant locations to the industrial plant. This surplus energy is used for the production of synthetic natural gas, which is then fed into a gas network. The gas network could be a public network used to transport the synthetic natural gas to consumers at distant sites. The gas network could be also a local network, i.e. a network serving only the industrial plant, an industrial zone or a residential area in close proximity to the industrial plant. Any type of gas network may include storage, e.g. means for underground storage, in case of excess of the synthetic natural gas production. Along with the synthetic natural gas, further oxygen and water are produced in the energy accumulator unit, which are stored locally in the industrial plant.

In the second operation mode, the oxygen and water stored on site are used for the production of electricity in the power plant unit of the industrial plant. The electricity generated in the power plant unit is fed into the public grid.

Thus, in the industrial plant surplus energy is used to produce synthetic natural gas (which is feed into a gas network) and oxygen, which oxygen later is used together with gas from the gas network to produce energy for the public grid.

In a preferred embodiment in the first operation mode the SNG is produced in a Sabatier reaction which involves the reaction of hydrogen with carbon dioxide at elevated temperatures (optimally 300-400° C.) and pressures in the presence of a catalyst to produce methane and water.

CO₂+4H₂→CH₄+2H₂O

The Sabatier reaction takes part in a Sabatier reactor which is part of the energy accumulator unit. Methanation is an important step in the creation of synthetic or substitute natural gas (SNG). The methane or SNG can be injected into the existing gas network, which has storage capacity of many months up to a couple of years.

In another preferred embodiment water from the water tank is used in an electrolysis reaction to produce oxygen and hydrogen, which hydrogen is used for the Sabatier reaction. Hence, the process of electrolysis of water is carried out in a preliminary step in order to create hydrogen for the Sabatier reaction. The oxygen, which is also generated in the water electrolysis, is stored in the oxygen tank of the industrial plant. In this case the water tank is not an external tank (even it might be). The water tank is accumulating and recycling the water received as product of methane burning.

In yet another preferred embodiment in the second operation mode the gas supplied from the gas network is burned together with oxygen from the oxygen tank in a combustor and the combustion mixture (containing carbon dioxide and steam) is used to drive a turbine. The combustor can be an external burner which is separated from the turbine or a burner integrated in the turbine.

Preferably, water from the water tank is used to regulate the temperature in the combustor by spraying the water into the mixture of gas and steam coming out of the combustor.

Still preferably, a steam-gas-mixture from the turbine is at least partially condensed in a heat exchanger producing water and steam and carbon dioxide mixture, where water and carbon dioxide are stored in the water tank and the carbon dioxide tank correspondingly.

In order to increase the efficiency of the process running in the industrial plant, in a preferred embodiment the heat from the heat exchanger is used for district heating or applied in a condensing turbine so that a turbine with increased the power is used.

Preferably, the energy accumulator unit and the power plant unit are operated alternatingly. The energy accumulator unit is operated in times of electricity surplus, while the power plant unit is operated in order to produce supplementary electricity on demand. Alternatively, the energy accumulator unit and the power plant unit are operated simultaneously.

### BRIEF DESCRIPTION OF THE DRAWINGS

Embodiments of the invention are now described, by way of example only, with reference to the accompanying drawing, of which the only FIGURE shows schematically an industrial plant 1.

### DETAILED DESCRIPTION OF INVENTION

The industrial plant 1 comprises an energy accumulator unit 3 for production of synthetic natural gas and a power plant unit 5 for production of electricity. In the power plant unit 5 additionally heat is produced. The industrial plant further comprises an oxygen tank 7, a water tank 9 and a carbon dioxide tank 11.

In the embodiment shown in the FIGURE the energy accumulator unit 3 consists of a hydrogen electrolysis system 13 and a Sabatier reactor 15. The hydrogen electrolysis system 13 is supplied with electricity from the public grid 17 by means of a power cable 19. The Sabatier reactor 15 is connected to a gas network via a gas discharge line 31.

The power plant unit 5 consists of a combustor 21, a turbine 23, a generator 25 and a heat exchanger 27. The combustor 21 is operated with gas from the gas network 29 provided by a gas supply line 33.

In a first operation mode of the industrial plant 1, in case of renewable electricity surplus in the public grid 17, this excessed energy is accumulated or stored in the energy accumulator unit 3 in form of oxygen stored in the oxygen tank 7 and SNG produced in the Sabatier reactor 15 and supplied to the gas network via the gas discharge line 31.

Water taken from the water tank 9 via a first water feed line 34 is provided to the hydrogen electrolysis system 13, in which the water molecules are split into hydrogen and oxygen using electrical current from the public grid 17. The oxygen stream is fed via an oxygen discharge line 35 into the oxygen tank 7 for storage purposes. The hydrogen stream is provided via a hydrogen feed line 37 into the Sabatier reactor 15, in which it reacts together with carbon dioxide provided by a feed line 39 from the carbon dioxide tank 11 to produce SNG (methane). The SNG produced in the energy accumulator unit 3 is fed into the gas network 29 by means of outflow gas pipe 31 and can be potentially transported to far distant underground gas storage facilities. Another product of the Sabatier reaction is water, which is discharged in a first water discharge line 41 and stored in the water tank 9.

Hence, in the first operation mode excessed energy from the public grid is stored locally by converting it into oxygen and water and the remaining part of the excessed energy is stored in form of synthetic natural gas in the gas network or stored in distant gas storage facilities connected to the gas network.

Normally, the power plant unit 5 is shutdown during the first operation mode.

In a second operation mode gas from the gas network 29 together with oxygen from the oxygen tank 7 and water from the water tank 9 are used in the power plant unit 5 to produce electricity. Natural gas or accumulated SNG is supplied from the gas network 29 via the gas supply line 33 into the combustor 21 to generate a steam-gas-mixture. The fuel is burned there with oxygen fed from the local oxygen tank 7 via an oxygen supply line 43. The flue gas of the combustion process is a mixture of water steam and carbon dioxide and its temperature is adjusted by the amount of water from the water tank 9 via a second water feed line 45 and injected into the combustor 21. The combustion mixture is fed into a turbine 23 where it expands.

The mechanical energy from the turbine 23 is transferred to the electrical generator 25, thus generating electricity. This electrical energy is conveyed to the public electricity grid 17.

The steam-gas-mixture is discharged from the turbine 23 into a heat exchanger 27. In the heat exchanger 27 the remaining thermal energy is transferred to a coolant 47 cycle. The steam-gas-mixture is cooled down there. The water steam from the mixture condenses and is discharged through a second water discharge line 49 into the water tank 9. The incondensable, clean carbon dioxide is discharged through a discharge line 51 into the local carbon dioxide tank 11.

This process, in which the locally stored energy (oxygen and water) together with SNG from the gas network are converted into electricity and heat, can be considered discharging of the energy accumulator unit 3. In this cycle, the total energy of methane, i.e. the upper heating value is employed compared with standard devices. This value is about 10% higher than the lower heating value.

Although the present invention has been described in detail with reference to the preferred embodiment, it is to be understood that the present invention is not limited by the disclosed examples, and that numerous additional modifications and variations could be made thereto by a person skilled in the art without departing from the scope of the invention.

1. A method for operation of an industrial plant, comprising an energy accumulator unit for production of synthetic natural gas, a power plant unit for production of electricity, an oxygen tank, a carbon dioxide tank, and a water tank, the method comprising:
in a first operation mode, supplying the energy accumulator unit with excessed electricity from a public grid in order to produce synthetic natural gas, wherein the produced synthetic natural gas is discharged in a gas network, while oxygen and water which are produced together with the synthetic natural gas are stored in the oxygen tank and the water tank correspondingly, and
in a second operation mode, using gas from the gas network together with oxygen from the oxygen tank and water from the water tank in the power plant unit to produce electricity, which is supplied to the public grid.

2. The method according to claim 1,
wherein in the first operation mode the SNG is produced in a Sabatier reaction.

3. The method according to claim 2,
wherein water from the water tank is used in an electrolysis reaction to produce oxygen and hydrogen, which hydrogen is used for the Sabatier reaction.

4. The method according to claim 1,
wherein in the second operation mode the gas supplied from the gas network is burned together with oxygen from the oxygen tank in a combustor and a resulting combustion mixture is used to drive a turbine.

5. The method according to claim 4,
wherein water from the water tank is used to regulate the temperature in the combustor.

6. The method according to claim 4,
wherein a steam-gas-mixture from the turbine is at least partially condensed in a heat exchanger producing water and steam and carbon dioxide mixture, where water and carbon dioxide are stored in the water tank and the carbon dioxide tank correspondingly.

7. The method according to claim 6,
wherein heat from the heat exchanger is used for district heating.

8. The method according to claim 1,
wherein the energy accumulator unit and the power plant unit are operated alternatingly.

9. An industrial plant adapted for carrying out the method according to claim 1, comprising:
an energy accumulator unit for production of synthetic natural gas,
a power plant unit for production of electricity,
an oxygen tank, a carbon dioxide tank and a water tank.
